(19) United States
(12) Patent Application Publication
(10) Pub. No.: US 2010/0076196 A1
(43) Pub. Date: Mar. 25, 2010

(54) PROCESS FOR THE PREPARATION OF ILOPERIDONE

(76) Inventor: Miklós Vértessy, Budapest (HU)

Correspondence Address: PATENT CENTRAL LLC;Stephan A. Pendorf, 1401 Hollywood Boulevard, Hollywood, FL, 33020, US

(21) Appl. No.: 12/555,066

(22) Filed: Sep. 8, 2009

Related U.S. Application Data
(60) Provisional application No. 61/098,404, filed on Sep. 19, 2008.

(51) Int. Cl. C07D 261/20

(52) U.S. Cl. 546/198

(57) ABSTRACT

A process for the preparation of crystalline 1-[4-[3-[4-(6-fluoro-1,2-benzisoxazol-3-yl)-1-piperidinyl]propoxy]-3-methoxyphenyl]-ethanone (Iloperidone), which comprises the reaction between 3-[1-(3-chloropropyl)-4-piperidinyl]-6-fluoro-1,2-benzisoxazole and 3-methoxy-4-hydroxy-acetophenone.

### FIELD OF THE INVENTION

The present invention relates to a new process for the preparation of crystalline 1-[4-[3-[4-(6-fluoro-1,2-benzisoxazol-3-yl)-1-piperidinyl]propoxy]-3-methoxyphenyl]-ethanone (Iloperidone).

The synthetic process comprises the reaction between 3-[1-(3-chloropropyl)-4-piperidinyl]-6-fluoro-1,2-benzisoxazole and 3-methoxy-4-hydroxy-acetophenone.

### BACKGROUND OF THE INVENTION

Iloperidone, whose chemical structure is shown bellow, is a neuroleptic and 5-hydroxytryptamine 2A antagonist to be used for the treatment of schizophrenia and general psychosis.

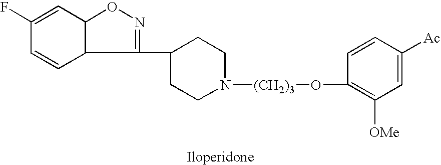

The product is protected by the U.S. Pat. No. 5,364,866, U.S. Pat. No. RE 39198 E and EP 402644 B1.

The first reported synthetic method for Iloperidone is described in patent EP 402644 A1.

In this document, the last step for the synthesis is the SN2 reaction between the nitrogen from the piperidine cycle and the halogen from the alkyl aryl ether, as is shown in Schema I.

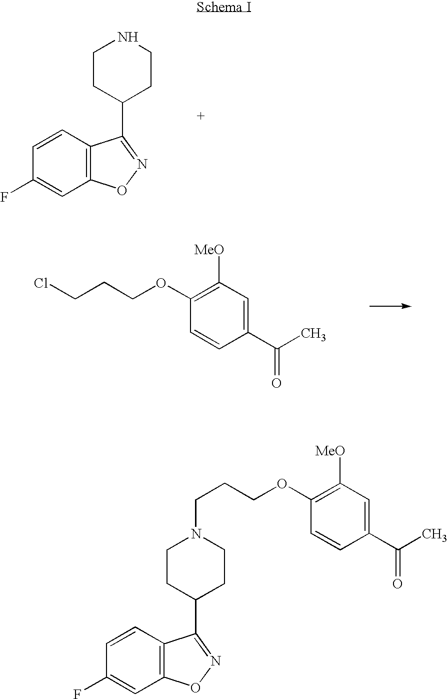

The yield of purified product obtained for this reaction is 58%.

Several patents were published after, describing essentially the same synthetic way such as U.S. Pat. No. 5,364,866 and U.S. Pat. No. 5,663,449.

### SUMMARY OF THE INVENTION

In accordance with the invention, a convenient manufacturing process is presented that has advantages over the known previous one.

The process is characterized by the reaction between 3-[1-(3-chloropropyl)-4-piperidinyl]-6-fluoro-1,2-benzisoxazole and 3-methoxy-4-hydroxy-acetophenone in an organic solvent or water in the presence of a base, as is shown in schema 2.

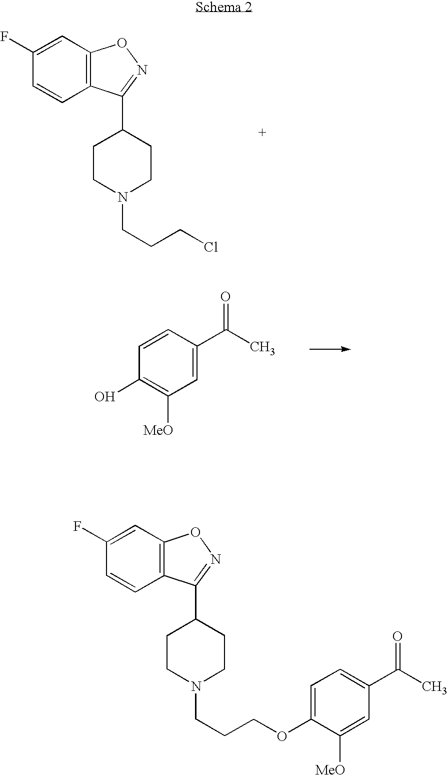

This is actually a Williamson reaction between a phenol, acting as a nucleophilic reagent and an alkyl chloride to yield the corresponding ether.

Besides the different substances involved into the reaction with regard to the published one, the present process shows some advantages.
Its yield (75%) is significantly higher than the published one (58%).
Obtained Iloperidone is colourless instead of beige.
It employs a friendlier and not so toxic solvent.
It employs an excess of a cheaper reagent (3-methoxy-4-hydroxy-acetophenone) instead of 1-[4-(2-chloroethoxy)-3-methoxyphenyl]ethanone.

### DESCRIPTION OF THE DRAWINGS

FIG. 1 shows an X-ray power diffractogram of crystalline Iloperidone.

### DETAILED DESCRIPTION OF THE INVENTION

Iloperidone is prepared by a novel and advantageous method.

In accordance with the method of the present invention, it is characterized by the reaction of 3-[1-(3-chloropropyl)-4-piperidinyl]-6-fluoro-1,2-benzisoxazole with 3-methoxy-4-hydroxy-acetophenone in the presence of a base.

The organic solvent(s) which may be used includes at least one solvent selected from the group consisting of tetrahydrofurane, dioxane, acetonitrile, water, toluene, methyl ethyl ketone, methyl isopropyl ketone, dimethylacetamide and dimethylformamide.

Methyl ethyl ketone, acetone and methyl isopropyl ketone are the most preferred.

The reaction of the present invention may be carried out at a temperature in the range of 20° C. to the boiling point of the solvent during 30 minutes to 24 hours, preferably 60 to 120° C. during 4 to 30 hours.

The base employed may be an organic or inorganic one, including one of the following: sodium hydroxide, carbonate or bicarbonate, potassium hydroxide carbonate or bicarbonate, lithium hydroxide, trimethyl amine, triethyl amine, pyridine, piperidine and DBU (1,8-diazabicyclo[5.4.0]undec-7-ene).

Potassium or sodium carbonates are the most preferred.

### EXAMPLES

The following examples are for illustrative purposes only and are not to be construed as limiting the invention. All temperatures are given in degrees Centigrade (° C.) unless indicated otherwise.

### Preparation of 1-[4-[3-[4-(6-fluoro-1,2-benzisoxazol-3-yl)-1-piperidinyl]propoxy]-3-methoxyphenyl]ethanone

### (A) Synthesis of 3-[1-(3-chloropropyl)-4-piperidinyl]-6-fluoro-1,2-benzisoxazole

In a 250 ml round bottomed flask equipped with a cool bath and magnetic stirrer, containing dimethylformamide (DMF) (180 ml); 3-(4-piperidinyl)-6-fluoro-1,2-benzisoxazole hydrochloride (10 g; 39 mM) and potassium carbonate (10.8 g; 77.9 mM) are added, giving rise to a suspension.

A solution of 1,3-bromochloropropane (6.0 ml; 61 mM) in DMF (7 ml) is dropped into the vigorously stirred suspension in a period of 30 minutes.

Stirring is continued for 17 hours at room temperature.

The resulting suspension is poured into water (120 ml) and extracted with ethyl acetate (120 ml).

The aqueous phase is extracted four additional times with 50 ml of ethyl acetate each time.

The joined organic phases are washed three times with a saturated sodium chloride solution and two additional times with water.

The washed organic phase is dried over sodium sulphate, filtered and the solvent eliminated under reduced pressure.

The oily residue, when treated with water (2.5 ml) yields, after half an hour, an off white crystalline solid.

After 1 hour in an ice bath, the suspension is filtered, washed with water and dried.

It yields 9.2 g (80%) of the desired product.

This crude product is employed as it is for the next step.

Anyway, a small portion of this crude product was purified by flash chromatography eluted with ethyl acetate.

The residue obtained from the best fractions, was crystallized from isopropanol to yield a colourless crystalline product.

Melting point: 69.2-70.2° C.

NMR (CDCl₃) δ 1.99 (m, 2H, ClCH₂CH₂CH₂), 2.03-2.21 (m, 6H, piper-3H, -5H, -2Hₐₓ, -6Hₐₓ,), 2.55 (t, J=7 Hz, 2H, ClCH₂CH₂CH₂), 3.02-3.11 (m, 3H, piper-2H_{eq}, -6H_{eq}, -4H), 3.64 (t, J=6.5 Hz, 2H, ClCH₂CH₂CH₂), 7.03-7.08 (m, 1H, benzisox-5H), 7.24 (dd, J=2 Hz and 8.5 Hz, 1H, benzisox-7H), 7.70 (dd, J=5 Hz and 8.5 Hz, 1H, benzisox-4H).

¹³C NMR (CDCl₃) δ 30.1 and 30.6 (ClCH₂CH₂CH₂, piper-3C and -5C), 34.6 (piper-4C), 43.3 (ClCH₂CH₂CH₂), 53.6 (piper-2C and -6C), 55.7 (ClCH₂CH₂CH₂), 97.5 (d, J=26 Hz, benzisox-7C), 112.3 (d, J=25 Hz, benzisox-5C), 117.3 (benzisox-3aC), 122.6 (d, J=11 Hz, benzisox-4C), 161.1 (benzisox-3C), 163.9 (d, J=14 Hz, benzisox-7aC), 164.1 (d, J=250 Hz, benzisox-6C)

IR (KBr) (cm⁻¹) Absorption at: 3046, 2824, 2776, 2743, 1615, 1514, 1499, 1472, 1447, 1416, 1379, 1352, 1271, 1256, 1235, 1123, 1030, 993, 980, 955, 893, 847, 814, 774, 640, 584, 530, 475 and 442.

### (B) Synthesis of 1-[4-[3-[4-(6-fluoro-1,2-benzisoxazol-3-yl)-1-piperidinyl]propoxy]-3-methoxyphenyl]-ethanone (Doper/done)

In a 150 ml round bottomed flask, equipped with a heating bath, a reflux condenser and magnetic stirring, containing methyl ethyl ketone (MEK) (45 ml), 4-hydroxy-3-methoxy acetophenone (3.36 g; 20.22 mM) and potassium carbonate (2.8 g; 20.22 mM) are added.

The obtained suspension is stirred and heated under reflux; 3-[1-(3-chloropropyl)-4-piperidinyl]-6-fluoro-1,2-benzisoxazole (5.0 g; 16.85 mM) is added and the suspension is heated under reflux (78-80° C.) overnight.

The reaction mixture is cooled at room temperature, and to the pale brown suspension, water (30 ml) is added giving rise to two phases.

The whole is evaporated under reduced pressure in order to eliminate MEK.

The resultant aqueous suspension is extracted with ethyl acetate (180 ml).

The organic phase is washed four times with a 10% sodium hydroxide solution (4×20 ml) and then twice with a saturated sodium chloride solution acidified with 10% (v/v) of HCL 0.1 N.

The organic phase is then washed with water (3×10 ml) and the solvent eliminated under reduced pressure.

A pale brown solid is obtained (6.65 g; 92.5%) which is crystallized from ethanol (9 ml).

It yields 5.84 g (81.3%) of beige crystals with melting point 114-119° C.

1.5 g of this product are recrystallized from ethanol (3.5 ml) giving 1.38 g (92%) of a colourless crystalline product with melting point 119.5-122° C.

IR (KBr) (cm⁻¹) Absorption at: 3033, 2950, 2822, 1669, 1615, 1594, 1586, 1511, 1462, 1449, 1416, 1381, 1314, 1264, 1221, 1179, 1150, 1125, 1078, 1044, 1032, 997, 986, 957, 886, 876, 853, 812, 781, 644, 612, 569, 475.

X-ray diffraction pattern expressed in terms of d-spacing (2θ), said diffraction pattern includes peaks at about 7.17, 10.18, 12.67, 14.37, 16.75, 17.13, 17.24, 17.60, 18.18, 18.31, 20.32, 20.41, 20.70, 21.60, 22.14, 23.64, 23.98, 26.40, 28.97, 30.78.

What is claimed is:

1. A procedure to prepare Iloperidone and salts thereof comprising the following steps:
a. reacting 3-[1-(3-chloropropyl)-4-piperidinyl]-6-fluoro-1,2-benzisoxazole with 3-methoxy-4-hydroxy-acetophenone in the presence of a base, using an organic solvent;
b. pouring the reaction medium into water;
c. extracting the aqueous phase with ethyl acetate and separating the layers;
d. washing the organic phase with aqueous sodium hydroxide solution, with acidified sodium chloride saturated solution and with water;
e. distilling off the solvent under reduced pressure;
f. crystallizing the product from an appropriate solvent;
g. recrystallizing the product from an appropriate solvent; and
h. preparing a solution of the free base into a solvent and adding the desired acid in the same solvent in order to obtain the corresponding salt.

2. The procedure according to claim 1, wherein the base used in step a is potassium carbonate or sodium carbonate.

3. The procedure according to claim 1, wherein the solvent used in step a is methyl ethyl ketone, methyl isopropyl ketone or acetone.

4. The procedure according to claim 1, wherein the molar ratio of the phenol to the alkyl halide in step a is from 0.9 to 1.4.

5. The procedure according to claim 1, wherein the temperature in step a is between 50° C. to 120° C.

6. The procedure according to claim 1, wherein the time of reaction for step a is between four and 30 hours.

7. The procedure according to claim 1, wherein the solvent of extraction in step c is ethyl acetate.

8. The procedure according to claim 1, wherein the solvent of crystallization in step f is ethanol or acetone.

9. The procedure according to claim 1, wherein the solvent of crystallization in step g is ethanol or acetone.

10. A crystalline form of 1-[4-[3-[4-(6-fluoro-1,2-benzisoxazol-3-yl)-1-piperidinyl]propoxy]-3-methoxyphenyl]ethanone (Iloperidone).

11. The crystalline form of Iloperidone of claim 10, having an X-ray powder diffraction pattern expressed in the terms of d-spacing (2θ); said diffraction pattern includes peaks at about 7.17, 10.18, 12.67, 14.37, 17.13, 17.24, 17.60, 20.32, 20.41, 20.70, 22.14, 23.64, 23.98, 26.40, 28.97, 30.78.

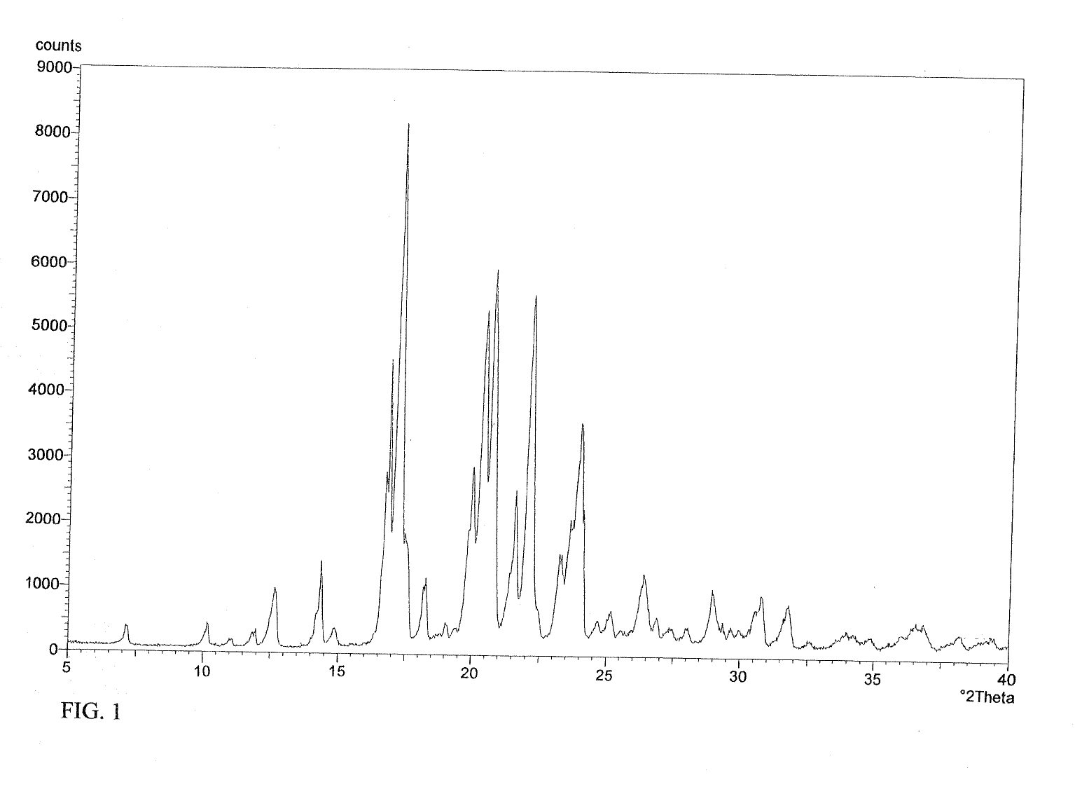

12. The crystalline form of Iloperidone of claim 10, wherein the X-ray powder diffraction pattern is substantially the same shown in FIG. 1.

13. The crystalline form of Iloperidone of claim 10, having an infrared spectrum that includes peaks at about 3033, 2950, 2822, 1669, 1615, 1594, 1586, 1511, 1462, 1449, 1416, 1264, 1221, 1150, 1125, 1032, 997, 986, 886, 853, 812, 781, 644, 612, 569 and 475 cm⁻¹.

14. A procedure to prepare Iloperidone and salts thereof comprising the following steps:
a. reacting 3-[1-(3-chloropropyl)-4-piperidinyl]-6-fluoro-1,2-benzisoxazole with 3-methoxy-4-hydroxy-acetophenone in the presence of a base, using an organic solvent, and
b. extracting the reaction product.
